(19) United States
(12) Patent Application Publication
(10) Pub. No.: US 2020/0294650 A1
(43) Pub. Date: Sep. 17, 2020

(54) COMPUTER, INFORMATION PROCESSING METHOD, AND NETWORK SYSTEM

(71) Applicant: SHARP KABUSHIKI KAISHA, Sakai City, Osaka (JP)

(72) Inventors: MASAYA UEDA, Sakai City (JP); YASUHIRO HARADA, Sakai City (JP); SHUHICHI HIRUKAWA, Sakai City (JP); AZUSA NAKANO, Sakai City (JP); CHIKAAKI KOGURE, Sakai City (JP); MASAYUKI NAKANO, Sakai City (JP); TAKAYUKI YUASA, Sakai City (JP)

(73) Assignees: SHARP KABUSHIKI KAISHA, Sakai City, Osaka (JP); SHARP KABUSHIKI KAISHA, Sakai City, Osaka (JP)

(21) Appl. No.: 16/083,946

(22) PCT Filed: Dec. 14, 2016
(86) PCT No.: PCT/JP2016/087210

§ 371 Date: Sep. 11, 2018

(30) Foreign Application Priority Data
Mar. 30, 2016 (JP) 2016-067446

(51) Int. Cl. G16H 20/70; G16H 20/30; A61B 5/16; A61B 10/00; G01N 33/50

(52) U.S. Cl. CPC G16H 20/70; G16H 20/30; G01N 2333/4742; A61B 10/00; G01N 33/50; A61B 5/165

(57) ABSTRACT

The purpose of the present disclosure is to provide a computer, an information processing method, and a network system by which information relating to a trend of a mental or physical state of a subject can be acquired more easily than before. Provided is a computer including: a memory configured to store first data obtained from a strand of hair acquired from a subject and second data obtained from a specimen acquired from the subject, the specimen being different in acquisition time from the strand of hair; and a processor configured to generate a trend of a mental or physical state of the subject based on the first data and the second data.

### TECHNICAL FIELD

The following disclosure relates to a technique for acquiring a mental or physical state of a subject, and more particular relates to a computer, an information processing method, and network system technology for acquiring information regarding the trend of a mental or physical state of a subject.

### BACKGROUND ART

Generally, techniques for obtaining the mental or physical state of a subject are known. For example, in JP 2004-213169 A (PTL 1), a health management system is disclosed. According to PTL 1, this health management system includes a management server, and users such as diabetes patients access the management server from a user terminal such as a personal computer via the Internet. In response to this access, the management server presents a questionnaire checklist with four categories of emotion, motivation (willingness), depression (low spirits), and stress on the user terminal. The user inputs responses to the questionnaire according to the implementation procedure for the checklist. The management server processes the response inputs and stores the results of this processing in a database. Accordingly, the user can access the database and learn the evaluation (questionnaire result) determined by the management server. However, in order to avoid presenting a questionnaire list of all the categories on the same day, the management server performs inquiries at timings deemed appropriate based on the priority level of each category.

In addition, in JP 2005-293130 A (PTL 2), a beauty salon support system is disclosed. According to PTL 2, a usage record storage unit that stores usage record information for customers is referenced, a status of a customer is obtained based on the usage record of the customer, proposal information corresponding to the status is extracted from a proposal information storage unit that stores proposal information for customers, and the proposal information is output.

Also, in NPL 1, Janet Yamada, “Hair Cortisol as a Potential Biologic Marker of Chronic Stress in Hospitalized Neonates,” Neonatology, KARGER issued on Mar. 14, 2007, it is described that cortisol in hair may be measured as a biomarker of newborn stress.

In addition, in NPL 2, Evan Russell et. al, “Hair cortisol as a biological marker of chronic stress Current status future directions and unanswered questions,” Psychoneuroendocrinology (2012) 37, 589-601, a technique for measuring cortisol in hair is disclosed.

### CITATION LIST

### Patent Literature

PTL 1: JP 2004-213169 A
PTL 2: JP 2005-293130 A

### Non Patent Literature

NPL 1: Janet Yamada, “Hair Cortisol as a Potential Biologic Marker of Chronic Stress in Hospitalized Neonates,” Neonatology, KARGER, Mar. 14, 2007.
NPL 2: Evan Russell et. al, “Hair cortisol as a biological marker of chronic stress Current status future directions and unanswered questions,” Psychoneuroendocrinology (2012) 37, 589-601.

### SUMMARY OF INVENTION

### Technical Problem

There is demand for a technique that makes it easier than before to acquire information regarding the trend of the mental or physical state of a subject. The present disclosure has been made to solve such problems, and has an object of providing a computer, an information processing method, and a network system capable of acquiring information relating to the trend of a mental or physical state of a subject more easily than before.

### Solution to Problem

According to an aspect of the invention, provided is a computer including: a memory configured to store first data obtained from a strand of hair acquired from a subject, and second data obtained from a specimen acquired from the subject, the specimen being different in acquisition time from the strand of hair; and a processor configured to generate a trend of a mental or physical state of the subject based on the first data and the second data.

Preferably, the computer further includes a communication interface. The processor acquires the first data and the second data from another device via the communication interface.

Preferably, the computer further includes a communication interface. The processor is configured to acquire the first data from a first other device via the communication interface and acquire the second data from a second other device via the communication interface.

Preferably, the second data is obtained from a strand of hair acquired from the subject, the strand of hair being different in acquisition time from the strand of hair.

Preferably, the second data is obtained from a specimen acquired from the subject other than a strand of hair, the specimen being different in acquisition time from the strand of hair.

Preferably, the processor is configured to generate a future state of the subject based on data acquired from a subject other than the subject.

Preferably, the processor is configured to generate advice for the future state.

Preferably, the processor is configured to: transmit, to the subject when a first predetermined condition is satisfied, information including the trend via the communication interface, and transmit, to a predetermined institution such as a police station, a prosecutor's office, a courthouse, or a hospital when a second predetermined condition is satisfied, information including the trend via the communication interface.

According to another aspect of the invention, an information processing method in a computer is provided. The information processing method includes: acquiring, by a processor, first data obtained from a strand of hair acquired from a subject; acquiring, by the processor, second data obtained from a specimen acquired from the subject, the specimen being different in acquisition time from the strand of hair; and generating, by the processor, a trend of a mental or physical state of the subject based on the first data and the second data.

According to still another aspect of the invention, provided is a network system including: a device configured to acquire first data obtained from a strand of hair acquired from a subject and second data obtained from a specimen acquired from the subject, the specimen being different in acquisition time from the strand of hair; and a computer configured to: receive the first data and the second data from the device, and generate a trend of a mental or physical state of the subject based on the first data and the second data.

According to yet another aspect of the invention, provided is a network system including: a first device configured to acquire first data obtained from a strand of hair acquired from a subject; a second device configured to acquire second data obtained from a specimen acquired from the subject, the specimen being different in acquisition time from the strand of hair; and a computer configured to: receive the first data from the first device, receive the second data from the second device, and generate a trend of a mental or physical state of the subject based on the first data and the second data.

### Advantageous Effects of Invention

As described above, according to the present disclosure, there is provided a computer, an information processing method, and a network system capable of acquiring information relating to the trend of a mental or physical state of a subject more easily than before.

### BRIEF DESCRIPTION OF DRAWINGS

FIG. 1 is an image diagram illustrating the overall configuration and operation overview of a network system 1 according to an embodiment 1.

FIGS. 2A to 2C are images illustrating merging of analysis result data of stress at a plurality of time periods, according to the embodiment 1.

FIG. 3 is a block diagram illustrating a hardware configuration of a server 100 according to the embodiment 1.

FIG. 4 is an image diagram illustrating a user database 121 according to the embodiment 1.

FIG. 5 is an image diagram illustrating an analysis database 122 according to the embodiment 1.

FIG. 6 is a flowchart illustrating information processing in the server 100, according to the embodiment 1.

FIG. 7 is a block diagram illustrating a hardware configuration of an analysis device 200, according to the embodiment 1.

FIG. 8 is a block diagram illustrating a hardware configuration of a smartphone 300 that serves as an electronic device according to the embodiment 1.

FIG. 9 is an image diagram illustrating the overall configuration and operation overview of the network system 1 according to an embodiment 2.

FIG. 10 is an image diagram illustrating the overall configuration and operation overview of the network system 1 according to an embodiment 3.

FIGS. 11A to 11D are images illustrating merging of analysis result data of stress at a plurality of time periods, according to the embodiment 3.

FIG. 12 is an image diagram illustrating the overall configuration and operation overview of the network system 1 according to an embodiment 4.

FIG. 13 is a flowchart illustrating information processing in the server 100, according to the embodiment 4.

FIG. 14 is an image diagram illustrating the user database 121 according to an embodiment 5.

FIG. 15 is an image diagram illustrating the analysis database 122 according to the embodiment 5.

FIG. 16 is an image diagram illustrating the overall configuration and operation overview of the network system 1 according to an embodiment 6.

FIG. 17 is a first image diagram illustrating the overall configuration and operation overview of the network system 1 according to an embodiment 7.

FIG. 18 is a second image diagram illustrating the overall configuration and operation overview of the network system 1 according to the embodiment 7.

FIG. 19 is an image diagram illustrating the overall configuration and operation overview of the network system 1 according to an embodiment 8.

FIG. 20 is an image diagram illustrating the overall configuration and operation overview of the network system 1 according to an embodiment 9.

FIG. 21 is an image diagram illustrating the user database 121 according to the embodiment 4.

### DESCRIPTION OF EMBODIMENTS

Herein, embodiments of the present disclosure will be described with reference to the drawings. In the following description, the same components are denoted by the same reference numerals. Their names and functions are also the same. Accordingly, a detailed description thereof will not be repeated.

### Embodiment 1

### Overall Configuration and Operation Overview of Network System

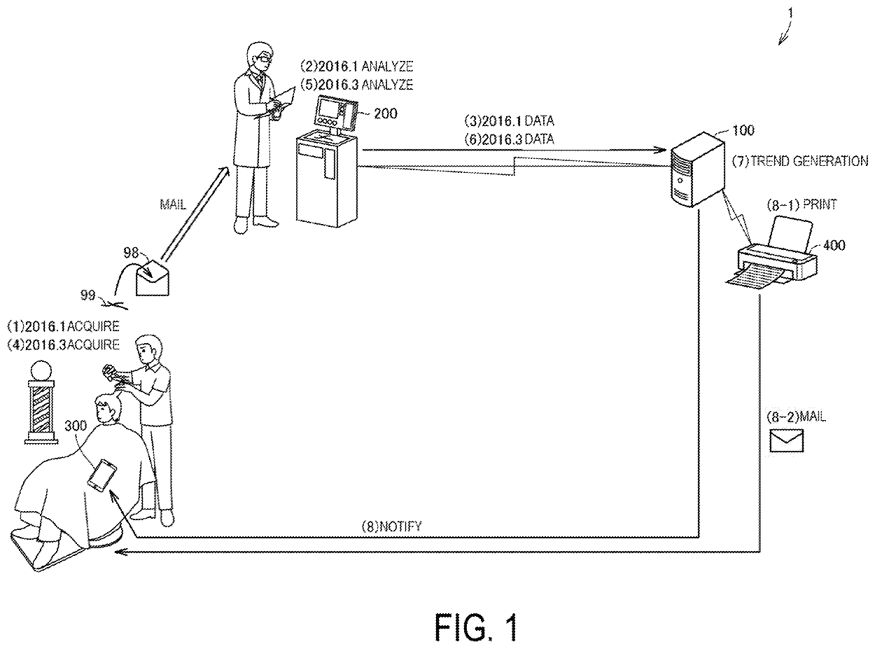

First, the overall configuration and operation overview of a network system 1 according to the present embodiment will be described with reference to FIG. 1. FIG. 1 is an image diagram illustrating the overall configuration and operation overview of the network system 1 according to an embodiment 1.

First, the overall configuration of the network system 1 according to the present embodiment will be described. The network system 1 according to the present embodiment primarily includes a server 100, an analysis device 200 capable of communicating with the server 100, a smartphone 300 that serves as a user terminal capable of communicating with the server 100, and a printer 400. Hereinafter, the operation overview of the network system 1 according to the present embodiment will be described.

(1) As an example, on Jan. 1, 2016, a user, who is a subject, visits a barber shop or a beauty salon. The barber acquires a strand of hair 99 from the subject that is cut at a predetermined distance from the hair root. The barber inserts the acquired strand of hair of the subject into an envelope 98, for example, and sends the envelope 98 to a laboratory, for example, by mail.

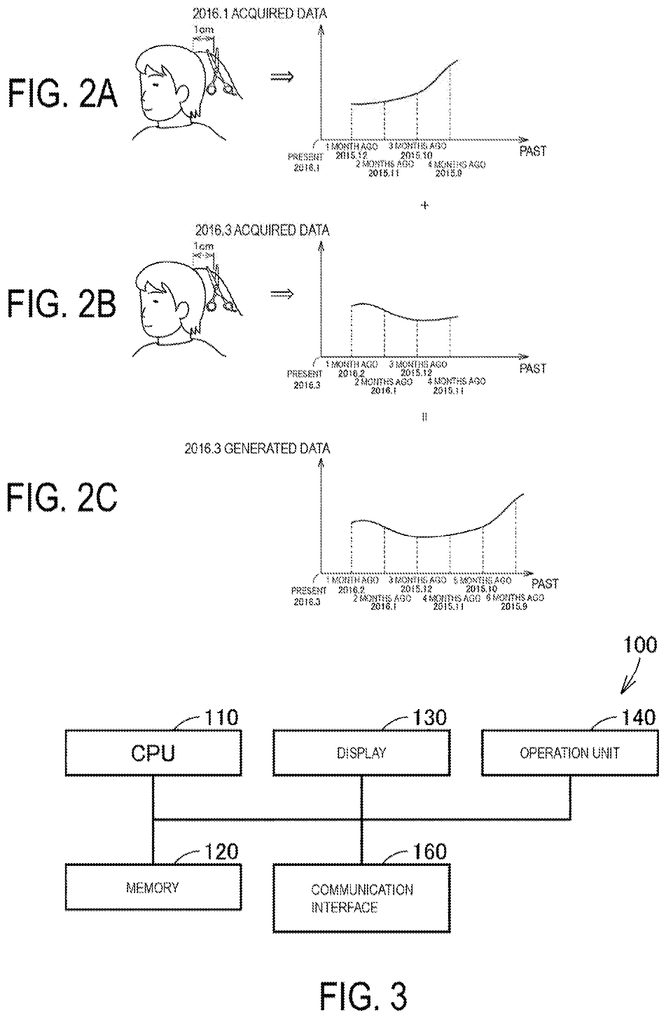

(2) At the laboratory, various analyses are performed on the received strand of hair. For example, as illustrated in FIG. 2A, the analysis device 200 examines the trend of numerical values indicating the magnitude of the stress of the subject by measuring the cortisol of each part of a strand of hair that was cut at a distance of 1 cm from the hair root. On average, as hair grows 1 cm per month, it is possible to acquire the trend of stress until approximately Dec. 1, 2015 from a strand of hair cut at a distance of 1 cm from the hair root. However, the object and method of the analyses by the analysis device 200 are not limited hereto.

(3) The analysis device 200 transmits data indicating the trend of the stress of the subject to the server 100. The server 100 stores the trend of past stress obtained from the strand of hair of the subject that was acquired on Jan. 1, 2016 in a database.

(4) Thereafter, the user who is the subject visits a barber shop or a beauty salon on Mar. 1, 2016, for example. The barber acquires a strand of hair from the subject that is cut at a predetermined distance from the hair root. The barber sends the acquired strand of hair of the subject to the laboratory by mail, for example.

(5) At the laboratory, the analysis device 200 performs various analyses on the received strand of hair. For example, as illustrated in FIG. 2B, the analysis device 200 examines the trend of numerical values indicating the magnitude of the stress of the subject by measuring the cortisol of each part of a strand of hair that was cut at a distance of 1 cm from the hair root. From the strand of hair cut at a distance of 1 cm from the hair root this time, it is possible to acquire the trend of stress until approximately Feb. 1, 2016. However, the object and method of the analyses by the analysis device 200 are not limited hereto.

(6) The analysis device 200 transmits data indicating the trend of the stress of the subject to the server 100. The server 100 stores the trend of past stress obtained from the strand of hair of the subject that was acquired on Mar. 1, 2016 in a database.

(7) Based on the trend of the stress acquired from the strand of hair of the subject that was acquired on Jan. 1, 2016 from the analysis device 200 and the trend of the stress acquired from the strand of hair of the same subject that was acquired on Mar. 1, 2016 from the analysis device 200, the server 100 combines these trends to generate a trend of the stress of the subject over a longer term. For example, as illustrated in FIG. 2C, based on the time period when the strand of hair was acquired the previous time and the time period when the strand of hair was acquired this time, the server 100 connects the two trends of the intensity of the stress to generate a trend of the stress intensity over a longer term.

(8) The server 100 transmits the generated data indicating the trend of stress over the long term to the smartphone 300 that serves as the user terminal of the subject. Alternatively, (8-1) the server 100 prints information indicating the generated trend of stress over the long term, and (8-2) a person responsible mails the printed material, for example, to the home of the subject.

In this way, subjects can more easily confirm trends of stress over a longer term than before.

Hereinafter, a specific configuration of the network system 1 for implementing such functionality will be described in detail.

Hardware Configuration of Server 100

First, one aspect of the hardware configuration of the server 100 that constitutes the network system 1 will be described. Note that FIG. 3 is a block diagram illustrating a hardware configuration of the server 100 according to the present embodiment.

With reference to FIG. 3, the server 100 includes a Central Processing Unit (CPU) 110, a memory 120, a display 130, an operation unit 140, and a communication interface 160 as primary components.

The CPU 110 controls each unit of the server 100 by executing a program stored in the memory 120. More particularly, the CPU 110 executes the various processes to be described below by executing a program stored in the memory 120 and referring to various data.

The memory 120 is realized by various Random Access Memories (RAM), various Read-Only Memories (ROM), flash memories, or the like. It should be noted that the memory 120 may also be realized by a storage medium or the like used through an interface, such as Universal Serial Bus (USB) (registered trademark) memory, a Compact Disc (CD), a Digital Versatile Disk (DVD), a Blu-ray (registered trademark) Disc (BD), a Solid State Drive (SD), a memory card, a hard disk, an Integrated Circuit (IC) card, an optical card, a mask ROM, an Erasable Programmable Read-Only Memory (EPROM), an Electronically Erasable Programmable Read-Only Memory (EEPROM) or the like.

The memory 120 stores programs to be executed by the CPU 110, data generated by execution of the programs by the CPU 110, input data, a database used for the services according to other embodiments, and the like. For example, the memory 120 stores a user database 121 and an analysis database 122. The memory 120 may store data indicating the analysis results indicating the state of a subject as illustrated in FIG. 2A or FIG. 2B, or may store data indicating a long-term trend generated from the analysis results indicating the state of the subject as illustrated in FIG. 2C.

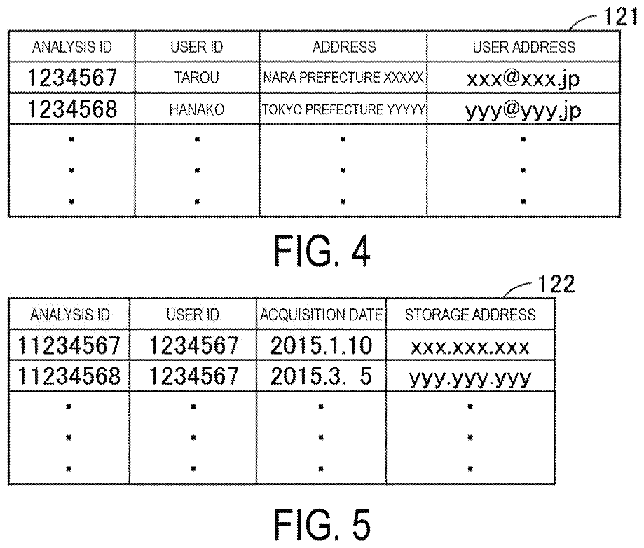

FIG. 4 is an image diagram illustrating the user database 121 according to the present embodiment. With reference to FIG. 4, the user database 121 stores, for each user, a user ID, a user name, an address, and a contract address of the user, for example. It should be noted that the user database 121 may additionally store telephone information, date of birth, gender, and the like for each user.

FIG. 5 is an image diagram illustrating an analysis database 122 according to the present embodiment. With reference to FIG. 5, the analysis database 122 stores, for each analysis, an analysis ID, a user ID, an acquisition date of a specimen or analysis result, and a storage address of analysis result data.

Returning to FIG. 3, the display 130 outputs text and images based on a signal from the CPU 110.

The operation unit 140 receives a command from a user and inputs the command to the CPU 110. Note that the display 130 and the operation unit 140 may constitute a touch panel.

The communication interface 160 transmits data from the CPU 110 to electronic devices via the Internet, a router, a base station, or the like. Conversely, the communication interface 160 receives data from electronic devices via the Internet, a router, a base station or the like, and delivers the data to the CPU 110.

### Information Processing in Server 100

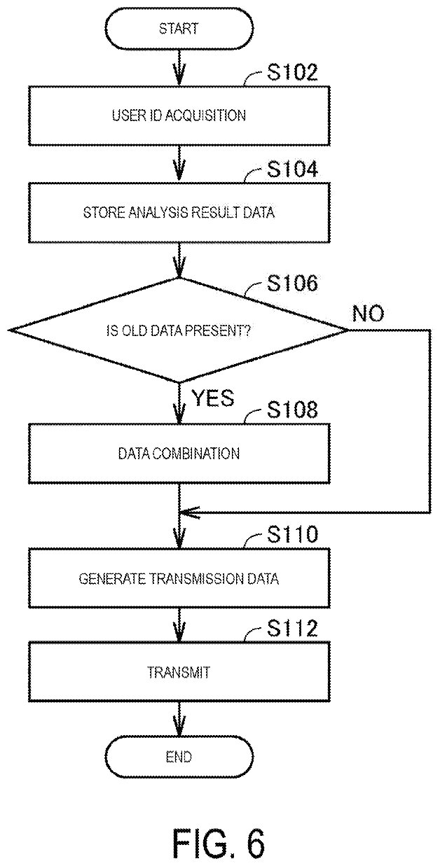

Next, information processing in the server 100 according to the present embodiment will be described with reference to FIG. 6. Note that FIG. 6 is a flowchart illustrating information processing in the server 100, according to the present embodiment.

Upon receiving data from the analysis device 200 via the communication interface 160, the CPU 110 of the server 100 executes the following processes. The CPU 110 acquires the user ID from the received data (Step S102).

The CPU 110 reads the analysis result data from the received data. The CPU 110 stores the analysis results in the memory 120, and stores the user ID and acquisition date in association with each other in the analysis database 122 (Step S104). It should be noted that although the analysis results described here differ depending on the service provided by the server 100, the analysis results are data indicating the mental or physical state of a subject with respect to one time, a plurality of times, or a predetermined period.

For example, in the case of a server 100 that provides a stress information provision service, as illustrated in FIG. 2A and FIG. 2B, information including a trend of the intensity of stress at a time period corresponding to the length from the hair root, which was generated from the amount of cortisol in each part of a strand of hair of a subject, may be acquired as the analysis results.

However, the CPU 110 may store the analysis data in a database of the memory of another device via the communication interface 160.

The CPU 110 references the analysis database 122 to determine whether or not past data for a target user is stored (Step S106). In the case that past data for the target user is not stored (NO in Step S106), the CPU 110 executes the processes starting from Step S110.

In the case that past data for the target user is stored (YES in Step S106), the CPU 110 combines the data acquired this time and the past data to generate long-term trend data (Step S108). For example, as illustrated in FIG. 2C, with respect to strands of hair obtained at multiple time periods from subjects having the same user ID, the CPU 110 generates information indicating the trend of stress over a longer term based on the time period when each of the plurality of strands of hair were collected, as well as the data indicating the trend of the stress intensity over a time period corresponding to the length from the hair root of the plurality of strands of hair.

The CPU 110 references the user database 121, and generates transmission data that includes information on the trend of the mental or physical state of the subject based on the analysis data received this time for the target user (Step S110). Alternatively, the CPU 110 references the user database 121, and generates transmission data that includes information on the long-term trend of the mental or physical state of the subject based on the trend data generated this time for the target user (Step S110).

The CPU 110 transmits the transmission data via the communication interface 160 (Step S112). The CPU 110 waits for the next data.

### Hardware Configuration of Analysis Device 200

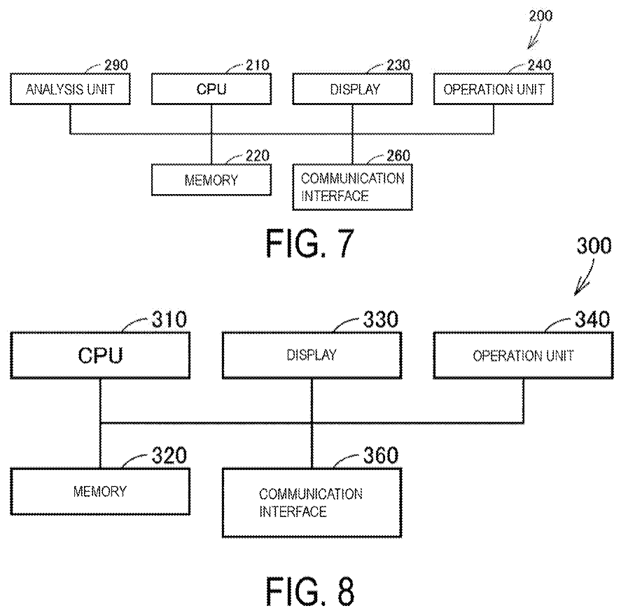

Next, one aspect of the hardware configuration of the analysis device 200 that constitutes the network system 1 will be described. Note that FIG. 7 is a block diagram illustrating a hardware configuration of the analysis device 200, according to the present embodiment.

With reference to FIG. 7, the analysis device 200 includes a CPU 210, a memory 220, a display 230, an operation unit 240, a communication interface 260, and an analysis unit 290 as primary components.

The CPU 210 controls each unit of the analysis device 200 by executing a program stored in the memory 220 or an external storage medium.

The memory 220 is realized by various RAMs, various ROMs, or the like. The memory 220 stores programs to be executed by the CPU 210, data generated by execution of the programs by the CPU 210, data received from the server 100, data input via the operation unit 240, and the like. For example, the memory 220 stores the analysis results in association with the user ID.

The display 230 outputs characters, images, and the like based on a signal from the CPU 110.

The operation unit 240 is realized by a button, a touch panel, a keyboard, or the like, receives a command from a user, and inputs this command to the CPU 210.

The communication interface 260 exchanges data with other devices by wired communication or wireless communication. For example, the CPU 210 transmits data indicating the analysis results obtained by the analysis unit 290, together with the user ID for this analysis and the date and time when the specimen was collected, to the server 100 via the communication interface 260.

The analysis unit 290 executes an analysis process of the specimen of the subject based on, for example, a signal from the CPU 210 or reception of a command from a person responsible via another route. For example, as illustrated in FIG. 2A and FIG. 2B, when measuring the stress of a subject, the analysis unit 290 measures the amount of cortisol in each part of a strand of hair. Then, based on this measurement result, the analysis unit 290 or the CPU 210 generates information including the trend of stress at a time period corresponding to the length from the hair root.

More particularly, based on the amount of cortisol in each part of the strand of hair, the analysis unit 290 measures the degree of stress of the subject at time periods corresponding to each part. Then, the measured values of the strand of hair at portions farther from the hair root are determined to be farther in the past from the time of cutting. If it is assumed that hair grows by 1 cm per month, stress values are obtained as follows. In the case that a strand of hair is cut at a location 1 cm from the hair root, the analysis unit 290 acquires stress values from one month before the time of cutting based on the amount of cortisol in the cut portion. The analysis unit 290 acquires stress values from two months before the time of cutting based on the amount of cortisol at a location 1 cm from the cut portion. The analysis unit 290 acquires stress values from three months before the time of cutting based on the amount of cortisol at a location 2 cm from the cut portion.

It should be noted that, in the case where a strand of hair is cut at a location 2 cm from the hair root, the analysis unit 290 acquires stress values from two months before the time of cutting based on the amount of cortisol in the cut portion. The analysis unit 290 acquires stress values from three months before the time of cutting based on the amount of cortisol at a location 1 cm from the cut portion. The analysis unit 290 acquires stress values from four months before the time of cutting based on the amount of cortisol at a location 2 cm from the cut portion.

### Hardware Configuration of Smartphone 300 as User Terminal

Next, one aspect of a hardware configuration in which the smartphone 300 serves as the user terminal that constitutes the network system 1 will be described. Note that FIG. 8 is a block diagram illustrating a hardware configuration of the smartphone 300 that serves as an electronic device according to the present embodiment. It should be noted that the user terminal is not limited to the smartphone 300, and may be a personal computer, a tablet terminal, a game machine, a wrist watch, or the like.

With reference to FIG. 8, the smartphone 300 includes a CPU 310, a memory 320, a display 330, an operation unit 340, and a communication interface 360 as primary components.

The CPU 310 controls each unit of the smartphone 300 by executing a program stored in the memory 320.

The memory 320 is realized by various RAMs, various ROMs, or the like. The memory 320 stores programs to be executed by the CPU 310, data generated by execution of the programs by the CPU 310, data received from the server 100, data input via the operation unit 340, information for identifying the user of the user terminal, and the like.

For example, the memory 320 stores identification information for the smartphone 300, and a user ID and password for the stress information provision service. In addition, the memory 320 stores the analysis results received by the CPU 310 via the communication interface 360.

The display 330 displays images and text based on data from the CPU 310. The operation unit 340 is constituted by a pointing device, a switch, or the like, and inputs various commands from a user to the CPU 310. Note that the user terminal may have a touch panel that includes the display 330 and the operation unit 340.

The communication interface 360 transmits data from the CPU 310 to the server 100 via the Internet, a router, or the like. Conversely, the communication interface 360 receives data from the server 100 via the Internet, a router, or the like, and delivers the data to the CPU 310.

### Embodiment 2

In the embodiment 1, the server 100 is configured to receive the same type of data for a plurality of time periods from one analysis device 200, and generate information indicating a long-term trend from the data. The present disclosure is not limited to such an embodiment, however.

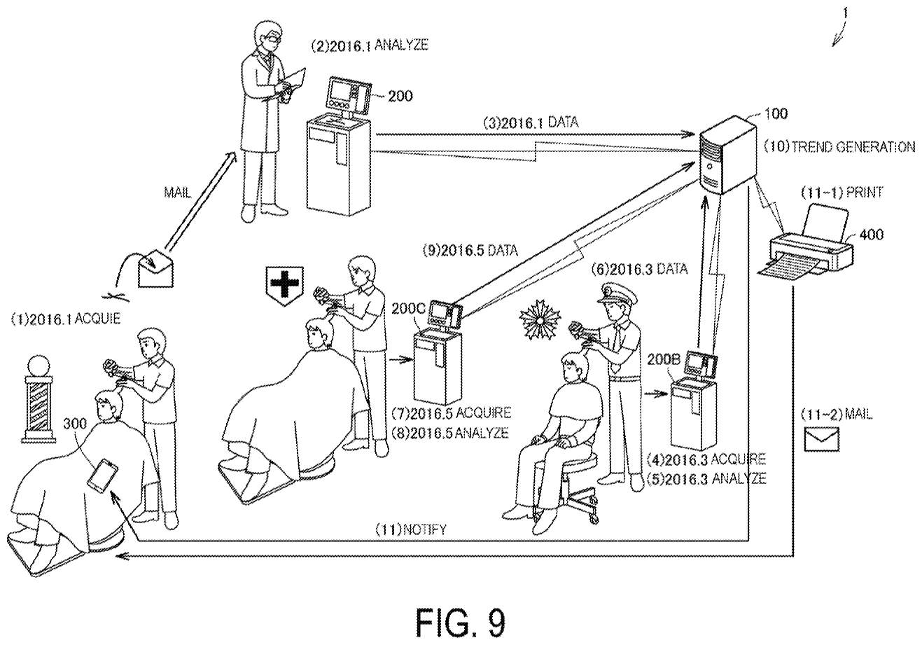

FIG. 9 is an image diagram illustrating the overall configuration and operation overview of the network system 1 according to the present embodiment. With reference to FIG. 9, the network system 1 according to the present embodiment primarily includes a server 100, a first analysis device 200 capable of communicating with the server 100, a second analysis device 200B capable of communicating with the server 100, a third analysis device 200C capable of communicating with the server 100, a smartphone 300 that serves as a user terminal capable of communicating with the server 100, and a printer 400.

(1) As an example, on Jan. 1, 2016, a user, who is a subject, visits a barber shop or a beauty salon. The barber acquires a strand of hair from the subject that is cut at a predetermined distance from the hair root. The barber sends the acquired strand of hair of the subject to a laboratory by mail, for example. (2) At the laboratory, various analyses are performed on the received strand of hair. (3) The analysis device 200 transmits data indicating the trend of the stress of the subject to the server 100. The server 100 stores the trend of past stress obtained from the strand of hair of the subject that was acquired on Jan. 1, 2016 in a database.

(4) Thereafter, if the user, who is the subject, goes to a police station on Mar. 1, 2016, for example, an officer may acquire a strand of hair of the subject cut at a predetermined distance from the hair root. (5) Using the second analysis device 200B, the officer performs various analyses on the received strand of hair. (6) The second analysis device 200B transmits data indicating the trend of the stress of the subject to the server 100. The server 100 stores the trend of past stress obtained from the strand of hair of the subject that was acquired on Mar. 1, 2016 in a database.

(7) Thereafter, if the user, who is the subject, goes to a hospital on May 1, 2016, for example, a person responsible may acquire a strand of hair of the subject cut at a predetermined distance from the hair root. (8) Using the third analysis device 200C, the person responsible performs various analyses on the received strand of hair. (9) The third analysis device 200C transmits data indicating the trend of the stress of the subject to the server 100. The server 100 stores the trend of past stress obtained from the strand of hair of the subject that was acquired on May 1, 2016 in a database.

(10) Based on the trend of the stress of the subject that was acquired on Jan. 1, 2016 from the analysis device 200, the trend of the stress of the same subject that was acquired on Mar. 1, 2016 from the second analysis device 200B, and the trend of the stress of the same subject that was acquired on May 1, 2016 from the third analysis device 200C, the server 100 combines these trends to generate a trend of the stress of the subject over a longer term.

(11) The server 100 transmits the generated data indicating the trend of stress over the long term to the smartphone 300 that serves as the user terminal of the subject. Alternatively, (11-1) the server 100 prints information indicating the generated trend of stress over the long term, and (11-2) the person responsible mails the printed material to, for example, the home of the subject.

In this way, subjects can more easily confirm trends of stress over a longer term than before.

Note that, as one aspect of the hardware configuration of the server 100 and the information processing in the server 100 are the same as those of the embodiment 1, a description thereof will not be repeated here. Naturally, in Step S102 of FIG. 6, the CPU 110 of the server 100 receives data from the analysis device 200, the second analysis device 200B, or the third analysis device 200C via the communication interface 160.

In addition, as one aspect of the hardware configuration of the analysis device 200 and one aspect of the hardware configuration of the smartphone 300 serving as the user terminal are the same as those of the embodiment 1, a description thereof will not be repeated here.

### Embodiment 3

In the embodiments 1 and 2, the server 100 is configured to receive the same type of data for a plurality of time periods from one or a plurality of analysis devices 200 to 200C, and generate information indicating a long-term trend from the data. The present disclosure is not limited to the embodiments, however.

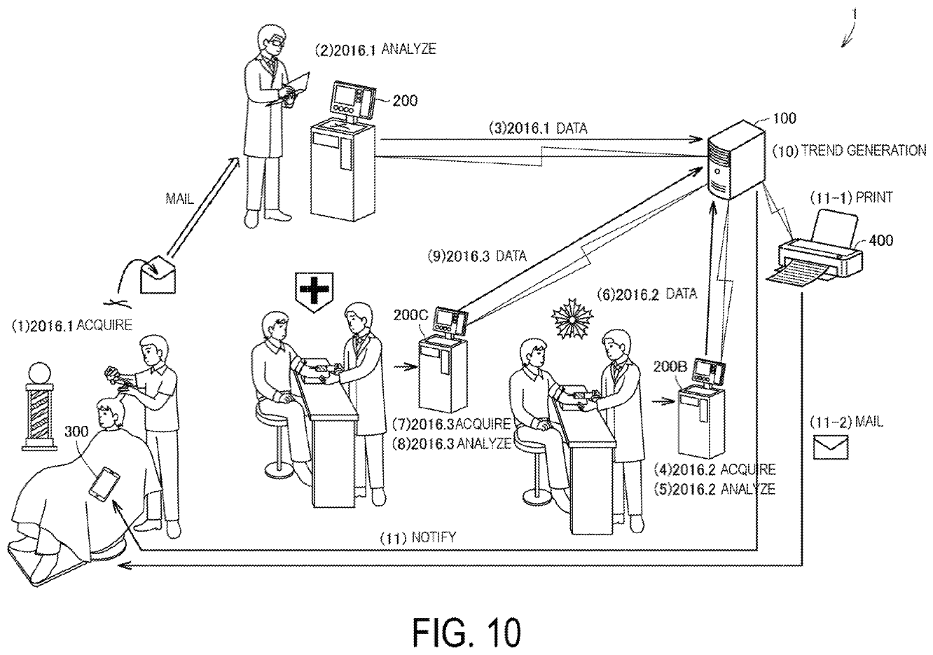

FIG. 10 is an image diagram illustrating the overall configuration and operation overview of the network system 1 according to the present embodiment. With reference to FIG. 10, the network system 1 according to the present embodiment primarily includes a server 100, a first analysis device 200 capable of communicating with the server 100, a second analysis device 200B capable of communicating with the server 100, a third analysis device 200C capable of communicating with the server 100, a smartphone 300 that serves as a user terminal capable of communicating with the server 100, and a printer 400.

Particularly in the present embodiment, at least one of the first analysis device 200, the second analysis device 200B, or the third analysis device 200C analyzes a specimen different from the other devices. Alternatively, at least one of the first analysis device 200, the second analysis device 200B, or the third analysis device 200C has the same specimen as an analysis target as the other devices, but the analysis method is different.

Note that, in the present embodiment, the analysis device 200 acquires the trend of past stress by analyzing the strand of hair. The second analysis device 200B and the third analysis device 200C acquire current stress values by analyzing blood, saliva, or the like.

Naturally, specimens analyzed by the analysis devices 200 to 200C are not limited to blood, saliva, or the like.

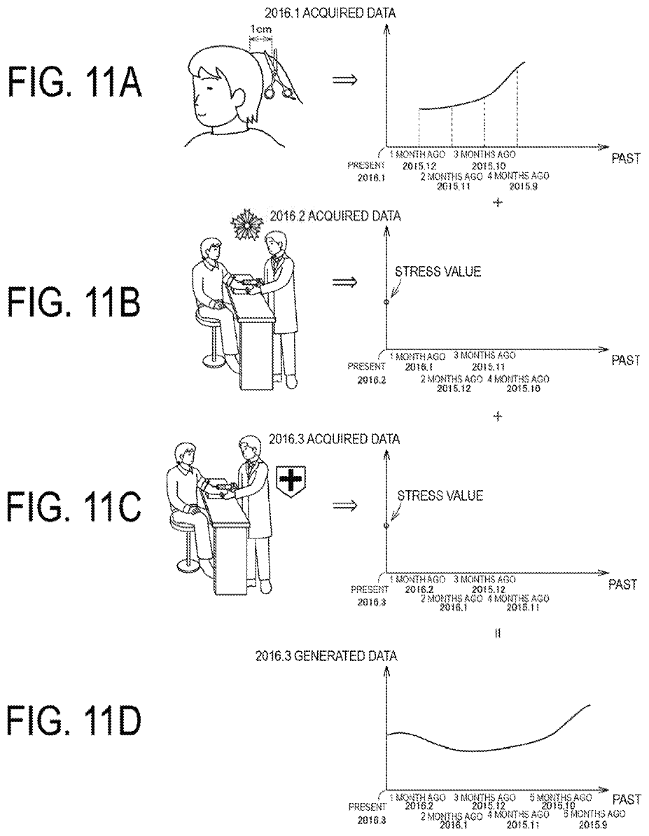

(1) As an example, on Jan. 1, 2016, a user, who is a subject, visits a barber shop or a beauty salon. The barber acquires a strand of hair from the subject that is cut at a predetermined distance from the hair root. The barber sends the acquired strand of hair of the subject to a laboratory by mail, for example. (2) At the laboratory, various analyses are performed on the received strand of hair. For example, as illustrated in FIG. 11A, the analysis device 200 examines the trend of a numerical value that indicates the magnitude of the stress of the subject by measuring the cortisol of each part of a strand of hair that was cut at a distance of 1 cm from the hair root. (3) The analysis device 200 transmits data indicating the trend of the stress of the subject to the server 100. The server 100 stores the trend of past stress obtained from the strand of hair of the subject that was acquired on Jan. 1, 2016 in a database.

(4) Thereafter, if the user, who is the subject, goes to a police station on Feb. 1, 2016, for example, an officer draws blood from the subject. (5) Using the second analysis device 200B, the officer performs various analyses on the blood. For example, as illustrated in FIG. 11B, the second analysis device 200B examines numerical values indicating the magnitude of the stress of the subject by measuring the cortisol of the blood. (6) The second analysis device 200B transmits data indicating the trend of the stress of the subject to the server 100. The server 100 stores the stress at that point in time obtained from the blood of the subject that was acquired on Feb. 1, 2016 in a database.

(7) Thereafter, if the user, who is the subject, goes to a hospital on Mar. 1, 2016, for example, a person responsible draws blood from the subject. (8) Using the third analysis device 200C, the person responsible performs various analyses on the blood. For example, as illustrated in FIG. 11C, the third analysis device 200C examines numerical values indicating the magnitude of the stress of the subject by measuring the cortisol of the blood. (9) The third analysis device 200C transmits data indicating the trend of the stress of the subject to the server 100. The server 100 stores the stress at that point in time obtained from the blood of the subject that was acquired on Mar. 1, 2016 in a database.

(10) Based on the trend of the stress of the subject that was acquired on Jan. 1, 2016 from the analysis device 200, the stress information for the same subject that was acquired on Feb. 1, 2016 from the second analysis device 200B, and the stress information for the same subject that was acquired on Mar. 1, 2016 from the third analysis device 200C, the server 100 combines these trends to generate a trend of the stress of the subject over a longer term. For example, as illustrated in FIG. 11D, based on the time period when the strand of hair was acquired and the time period when the blood was drawn, the server 100 connects the multiple stress trends or stress values to generate a trend of the stress over a longer term.

(11) The server 100 transmits the generated data indicating the trend of stress over the long term to the smartphone 300 that serves as the user terminal of the subject. Alternatively, (11-1) the server 100 prints information indicating the generated trend of stress over the long term, and (11-2) the person responsible mails the printed material to, for example, the home of the subject.

In this way, subjects can more easily confirm trends of stress over a longer term than before.

Note that, as one aspect of the hardware configuration of the server 100, the information processing in the server 100, one aspect of the hardware configuration of the analysis device 200, and one aspect of the hardware configuration of the smartphone 300 that serves as the user terminal are the same as those of the embodiments 1 and 2, a description thereof will not be repeated here.

### Embodiment 4

In the embodiments 1 to 3, the server 100 is configured to receive the same or different types of data for a plurality of time periods from one or a plurality of analysis devices 200 to 200C, and generate information indicating a long-term trend from the data. The present disclosure is not limited to the embodiments, however.

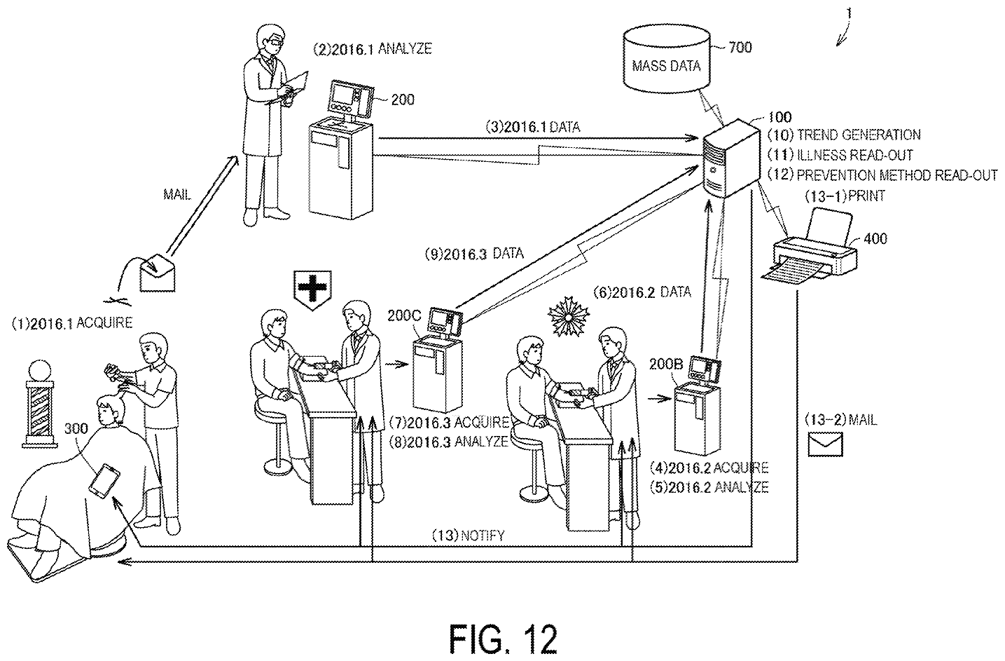

FIG. 12 is an image diagram illustrating the overall configuration and operation overview of the network system 1 according to the present embodiment. With reference to FIG. 12, the network system 1 according to the present embodiment primarily includes a server 100, a first analysis device 200 capable of communicating with the server 100, a second analysis device 200B capable of communicating with the server 100, a third analysis device 200C capable of communicating with the server 100, a smartphone 300 that serves as a user terminal capable of communicating with the server 100, a printer 400, and a database 700.

In the present embodiment, data regarding trends of the physical or mental states of a plurality of subjects, illness data corresponding to these trends, advice data such as prevention methods corresponding to these illnesses, and the like are stored in a database 700 that stores mass data. However, the mass data stored in the database 700 may be stored in other devices such as the server 100. In addition, the mass data is not limited to an embodiment in which the mass data is stored in a single computer, but may also be stored in the cloud, for example.

(1) to (3) For example, the analysis device 200 transmits data indicating the trend of the stress of the subject to the server 100. The server 100 stores the trend of past stress obtained from the strand of hair of the subject that was acquired on Jan. 1, 2016 in a database.

(4) to (6) Thereafter, the second analysis device 200B transmits data indicating the stress of the subject to the server 100, for example. The server 100 stores the stress at that point in time obtained from the blood of the subject that was acquired on Feb. 1, 2016 in a database.

(7) to (9) Subsequently, the third analysis device 200C transmits data indicating the stress of the subject to the server 100. The server 100 stores the stress at that point in time obtained from the blood of the subject that was acquired on Mar. 1, 2016 in a database.

(10) Based on the trend of the stress of the subject that was acquired on Jan. 1, 2016 from the analysis device 200, the stress information for the same subject that was acquired on Feb. 1, 2016 from the second analysis device 200B, and the stress information for the same subject that was acquired on Mar. 1, 2016 from the third analysis device 200C, the server 100 combines these trends to generate a trend of the stress of the subject over a longer term.

(11) Based on the acquired trends of the physical or mental state of the subject, the server 100 acquires, for example, data on illnesses corresponding to these trends by referencing the database 700.

(12) By referencing the database 700, the server 100 acquires advice data and the like such as prevention methods corresponding to the illness data.

(13) The server 100 transmits generated data indicating the trend of stress over the long-term, data indicating illnesses with a high contraction likelihood, and data indicating advice such as prevention methods of these illnesses to the smartphone 300 that serves as a user terminal of the subject. Alternatively, (13-1) the server 100 prints this information, and (13-2) a person responsible mails the printed material to, for example, the home of the subject.

In this way, subjects can more easily confirm trends of stress over a longer term than before.

Note that, as one aspect of the hardware configuration of the server 100, one aspect of the hardware configuration of the analysis device 200, and one aspect of the hardware configuration of the smartphone 300 that serves as the user terminal are the same as those of the embodiments 1 and 2, a description thereof will not be repeated here.

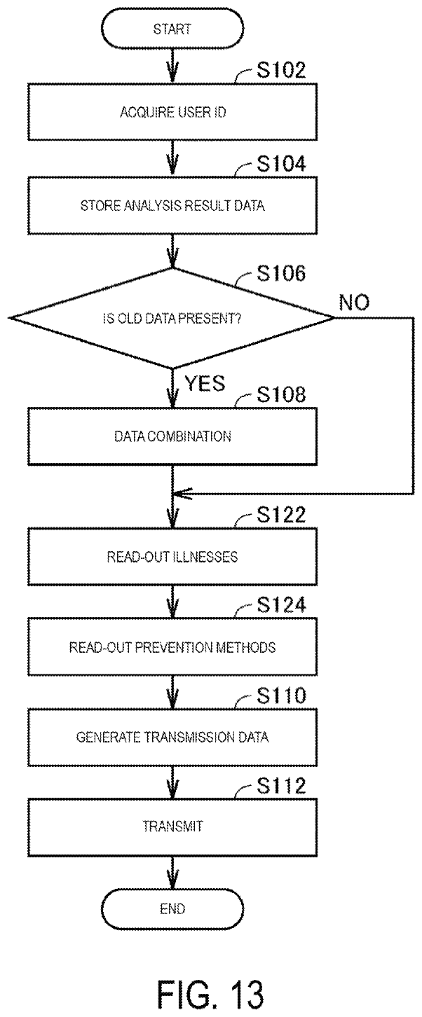

In the following, information processing in the server 100 according to the present embodiment will be described in more detail with reference to FIG. 13. Note that FIG. 13 is a flowchart illustrating information processing in the server 100, according to the present embodiment.

Upon receiving data from the analysis devices 200 to 200C via the communication interface 160, the CPU 110 of the server 100 executes the following processes. The CPU 110 acquires a user ID from the received data (Step S102).

The CPU 110 reads the analysis result data from the received data. The CPU 110 stores the analysis results in the memory 120, and stores the user ID and acquisition date in association with each other in the analysis database 122 (Step S104). It should be noted that although the analysis results described here differ depending on the service provided by the server 100, the analysis results are data indicating the mental or physical state of a subject with respect to one time, a plurality of times, or a predetermined period.

However, the CPU 110 may store the analysis data in a database of the memory of another device via the communication interface 160.

The CPU 110 references the analysis database 122 to determine whether or not past data for a target user is stored (Step S106). In the case that past data for the target user is not stored (NO in Step S106), the CPU 110 executes the processes starting from Step S110.

In the case that past data for the target user is stored (YES in Step S106), the CPU 110 combines the data acquired this time and the past data to generate long-term trend data (Step S108).

The CPU 110 references the database 700 and reads data regarding illnesses corresponding to the long-term trend data (Step S122). For example, the CPU 110 matches long-term trend data generated this time with a plurality of long-term trend data stored in the database 700, and identifies illnesses corresponding to data having a high degree of conformance. The CPU 110 reads advice data and the like such as prevention methods corresponding to the illnesses (Step S124).

The CPU 110 references the user database 121, and generates transmission data that includes information regarding the trends of the mental or physical state of the subject, information regarding illnesses for which the subject has a high contraction likelihood, and information regarding advice such as prevention methods with respect to these illnesses (Step S110).

The CPU 110 transmits the transmission data via the communication interface 160 (Step S112). The CPU 110 waits for the next data.

It should be noted that, in cases where predetermined conditions regarding the attributes of the user acting as the subject, attributes of the user to whom the information is notified, the type of the illnesses, the type of advice, or the like are satisfied, the CPU 110 may transmit the various types of information mentioned above.

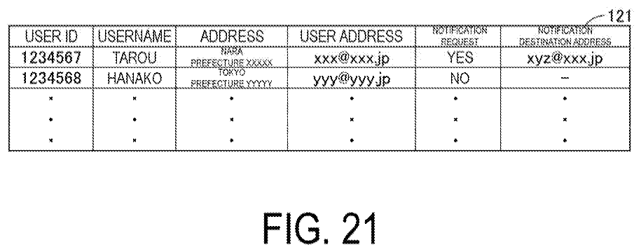

For example, the CPU 110 may receive information from the subject, their family, or the like indicating whether or not information notifications are requested or information indicating whether or not information notifications are permitted, and may store these designations in the user database 121 in association with the user ID of the subject. Alternatively, the CPU 110 may receive information regarding the information notification destination from the subject, their family, or the like, and may store this designation in the user database 121 in association with the user ID of the subject. Put differently, as illustrated in FIG. 21, the user database 121 according to the present embodiment stores a presence/absence for notification permissions, notification destination addresses, and the like for each user.

Then, as in the following Table 1, according to determination criteria based on the consenter of the analysis result notification and the analysis result notification destination, the CPU 110 references the user database 121, determines whether or not to transmit the transmission data, transmits the transmission data to the subject when a first condition is satisfied, and transmits the transmission data to a predetermined institution such as a police station, a prosecutor's office, a courthouse, a hospital, or the like when a second condition is satisfied.

TABLE 1

| | | Notification Destination of Result | |
|---|---|---|---|
| | | | B. Notification to Person other |
| | | A. Notification to Subject | than Subject |
| Person who | Subject | Intention declaration or consent | Intention declaration or |
| wishes | | from subject is required | consent from subject is |
| for/consents | | | required |
| to analysis | Person other than | When consent of the subject and | assuming consent from the |
| result | subject (Note 1) | a request from a person other | subject, |
| notifications | | than the subject are received, | send notification to the person |
| | | | other than subject based on |
| | | | the request from the person |
| | | | other than the subject. (Note 2, 3) |

(Note 1)
1. Doctors, hospitals, child consultation centers, specialists such as police officer and prosecutor, beauty salons, and 2. remote family members can be considered.
(Note 2)
However, these do not apply when there are special circumstances as the following.
In cases where the subject is a minor, has dementia or the like, or otherwise cannot make decisions for him/herself. (Possible only by the determination of a guardian)
Mental assessment or evidence examination by police, prosecution, judicial system, or the like. (Consent of the subject is unnecessary)
(Note 3)
Providing information to doctors (such as psychiatrists), or guardianship by remote family members are considered.

### Embodiment 5

In the embodiments 1 to 4, the server 100 is configured to identify the subject of data from one or a plurality of analysis devices 200 to 200C based on a user ID. The present disclosure is not limited to the embodiments, however. For example, a subject may be identified using a public ID such as a My Number, a license number, a passport number, or the like instead of the user ID.

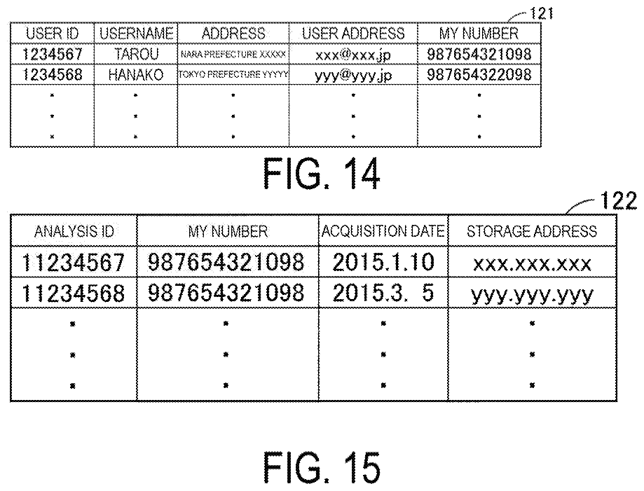

For example, the memory 120 of the server 100 stores the user database 121 of FIG. 14 and the analysis database 122 of FIG. 15 in place of the user database 121 of FIG. 4 and the analysis database 122 of FIG. 5.

More particularly, FIG. 14 is an image diagram illustrating the user database 121 according to the present embodiment. With reference to FIG. 14, the user database 121 stores, for each user, a user ID, a user name, an address, a contact address of the user, and a public ID. Embodiments in which the user ID itself is not used are also possible.

FIG. 15 is an image diagram illustrating the analysis database 122 according to the present embodiment. With reference to FIG. 5, the analysis database 122 stores, for each analysis, an analysis ID, a public ID, an acquisition date, and a storage address.

In this way, the server 100, the analysis devices 200 to 200C, and the smartphone 300 can use the public ID and exchange data to identify the subjects. For example, upon receiving data from the analysis devices 200 to 200C via the communication interface 160, the CPU 110 of the server 100 reads a public ID from the received data (Step S102). Also, the CPU 210 of the analysis devices 200 to 200C transmits data indicating the analysis results, the public ID of the subject, and the acquisition date and time to the server 100, for example.

Note that, as the overall configuration and operation overview of the network system 1, one aspect of the hardware configuration of the server 100, the information processing in the server 100, one aspect of the hardware configuration of the analysis device 200, and one aspect of the hardware configuration of the smartphone 300 that serves as the user terminal are the same as those of the embodiments 1 to 3, a description thereof will not be repeated here.

### Embodiment 6

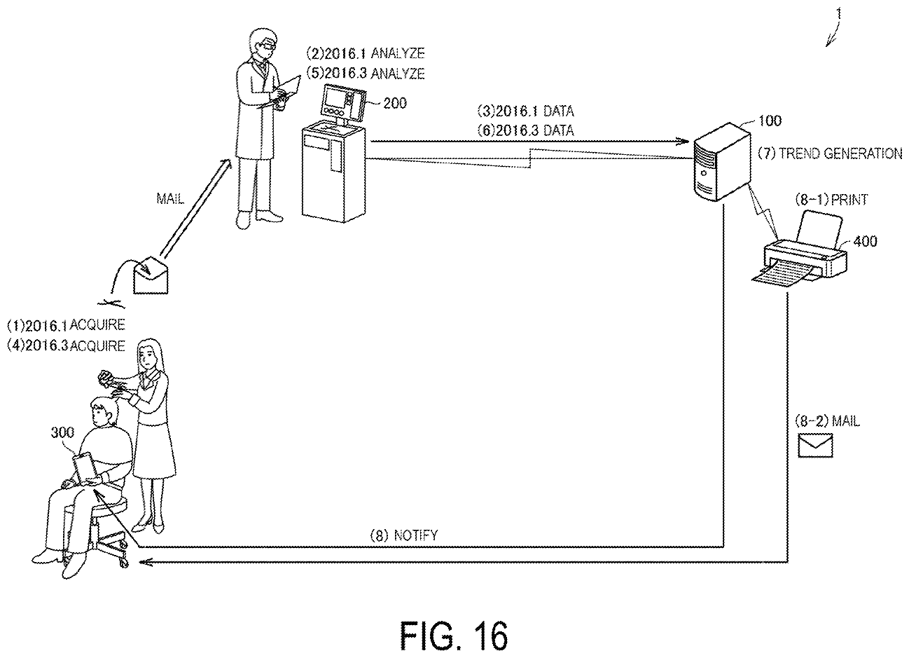

In the embodiments 1 to 5, a business model for cutting hair in a barber shop or beauty salon is included. The present disclosure is not limited to the embodiments, however. For example, as illustrated in FIG. 16, a business model may also be utilized in which a user, who is a subject, has a colleague, a friend or the like cut his or her hair at a predetermined location from the hair root at an office, at home, or the like, and mails this strand of hair to an analysis center. It should be noted that, naturally, a user who is a subject may cut their hair by themselves.

### Embodiment 7

In the embodiments 1 to 6, the analysis device 200 is disposed at a place different from the barber shop or beauty salon, and also at a different place from the server 100, such as at an analysis center, for example. The present disclosure is not limited to the embodiments, however.

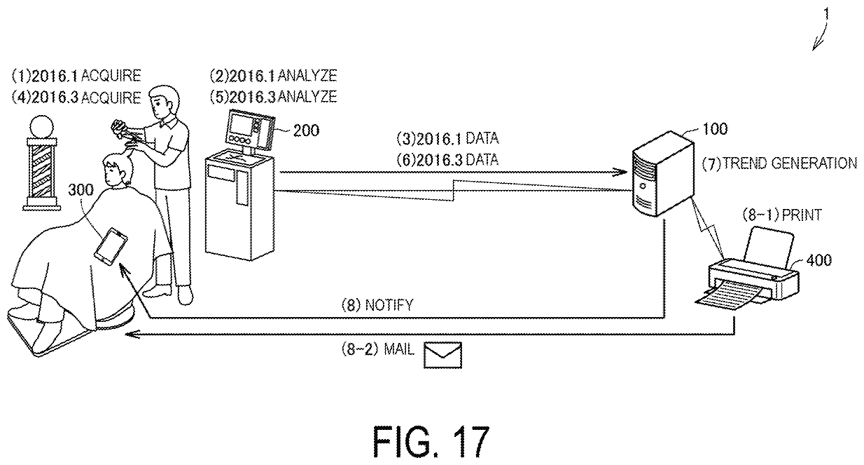

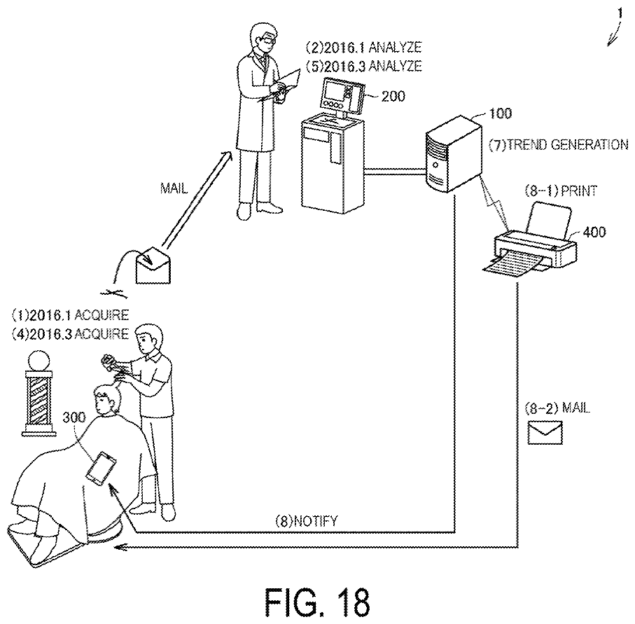

For example, as illustrated in FIG. 17, the analysis device 200 may be disposed in a barber shop or a beauty salon. Alternatively, the analysis device 200 is not limited to police stations and hospitals, but may be located in other places such as offices, nursing homes, schools, fitness gyms, game centers, and the like. Alternatively, as illustrated in FIG. 18, for example, the analysis device 200 may be disposed in the same place as the server 100, such as in the same building as the server 100 or the same room as the server 100.

### Embodiment 8

In the embodiments 1 to 7, although the business model includes the analysis device 200 and corresponding steps for analyzing specimens from barber shops, beauty salons, or both, the present disclosure is not limited to the embodiments.

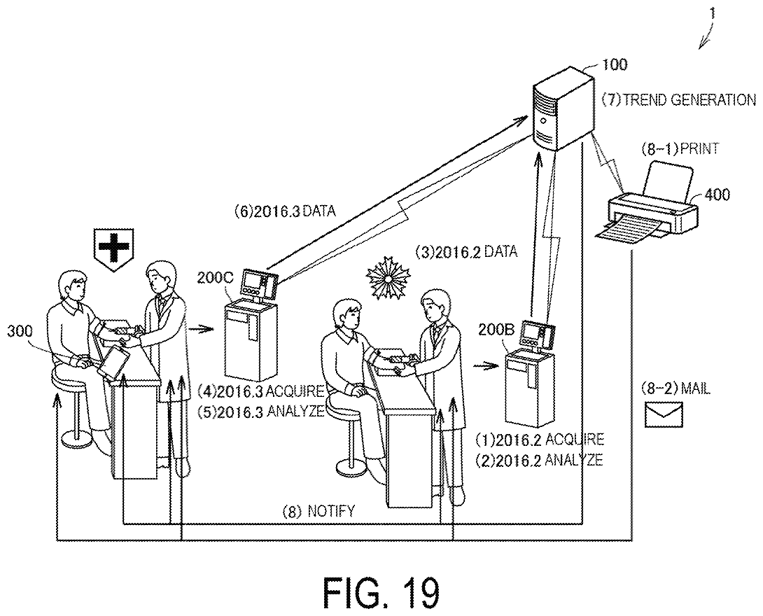

For example, as illustrated in FIG. 19, a barber shop or a beauty salon need not be included in the network system 1 in the first place. Also, naturally, the network system 1 need not include the analysis device 200 for analyzing specimens sent by mail from barber shops, beauty salons, or offices.

### Embodiment 9

In the embodiments 1 to 8, the network system 1 includes the server 100, the analysis devices 200 to 200C, and the smartphone 300. However, the roles of all or a portion of one of these devices may be shared by a plurality of devices, or one device may carry out all or a part of the roles of this plurality of devices.

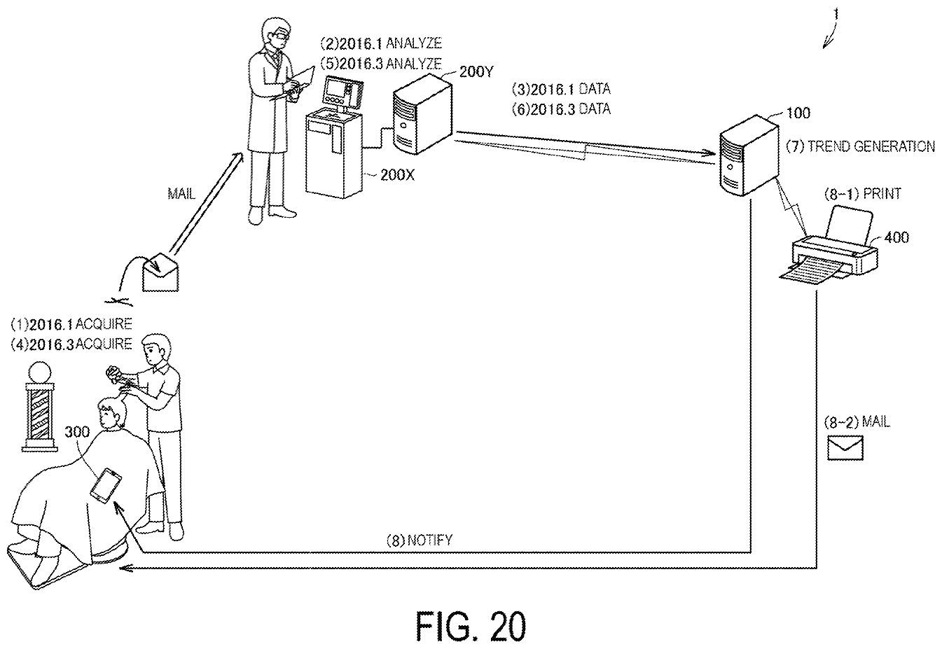

For example, as illustrated in FIG. 20, the analysis device 200 may be constituted by an analysis device 200X that primarily performs analysis, and a server 200Y that collects the analysis results and transmits the analysis results to the server 100.

Conversely, the analysis device 200 may carry out part of the role of the server 100, or the server 100 may carry out part of the role of the analysis device 200.

### OTHER APPLICATION EXAMPLES

It is needless to say that the present disclosure can also be applied to cases where the above-described embodiments are achieved by supplying a program to a system or a device. Also, it is possible to attain the effects of the present disclosure by supplying a storage medium (or a memory) that stores a program implemented as software for achieving the present disclosure to a system or a device, and having a computer (or CPU or MPU) of the system or device read and execute the program code stored in the storage medium.

In this case, the program code read from the storage medium implements the functions of the above-described embodiments, and the storage medium that stores this program code constitutes the present disclosure.

In addition, it is needless to say that by executing the program code read by the computer, not only are the functions of the above-described embodiments realized, but cases in which an operating system (OS) or the like operating on a computer performs part of or all of the actual processing based on the instructions of the program code and the functions of the above-described embodiments are realized by this processing are also included.

Further, it is needless to say that cases are also included in which, after the program code read out from the storage medium is written in another storage medium provided in a function expansion board inserted in the computer or in a function expansion unit connected to the computer, a CPU or the like provided in the function expansion board or function expansion unit performs part of or all of the actual processing based on the instructions of the program code and the functions of the above-described embodiments are realized by this processing.

### SUMMARY

In the embodiments 1 to 9 described above, provided is a computer 100 including: a memory 120 configured to store first data obtained from a strand of hair acquired from a subject and second data obtained from a specimen acquired from the subject at a different acquisition time from that of the strand of hair; and a processor 110 configured to generate a trend of a mental or physical state of the subject based on the first data and the second data.

Preferably, the computer 100 further includes a communication interface 160. The processor 110 acquires the first data and the second data from the other device 200 via the communication interface 160.

Preferably, the computer 100 further includes a communication interface 160. The processor 110 is configured to acquire the first data from a first other device 200, 200Y via the communication interface 160, and acquire the second data from a second other device 200B, 200C via the communication interface 160.

Preferably, the second data is obtained from a strand of hair acquired from the subject at a different acquisition time from that of the strand of hair.

Preferably, the second data is obtained from a specimen acquired from the subject other than a strand of hair at a different acquisition time from that of the strand of hair.

Preferably, the processor 110 is configured to generate a future state of the subject based on data 700 acquired from a subject other than the subject.

Preferably, the processor 110 is configured to generate advice for the future state.

Preferably, the processor 110 is configured to: transmit, to the subject when a first predetermined condition is satisfied, information including the trend via the communication interface 160, and transmit, to a predetermined institution such as a police station, a prosecutor's office, a courthouse, or a hospital when a second predetermined condition is satisfied, information including the trend via the communication interface 160.

In the embodiments 1 to 9 described above, an information processing method in the computer 100 is provided. The information processing method includes: acquiring, by a processor 110, first data obtained from a strand of hair acquired from a subject; acquiring, by the processor 110, second data obtained from a specimen acquired from the subject at a different acquisition time from that of the strand of hair; and generating, by the processor 110, a trend of a mental or physical state of the subject based on the first data and the second data.

In the embodiments 1 to 9 described above, provided is a network system 1 including: a device 200 configured to acquire first data obtained from a strand of hair acquired from a subject and second data obtained from a specimen acquired from the subject at a different acquisition time from that of the strand of hair; and a computer 100 configured to: receive the first data and the second data from the device 200, and generate a trend of a mental or physical state of the subject based on the first data and the second data.

In the embodiments 2 to 9 described above, a network system 1 is provided, the network system 1 including: a first device 200 configured to acquire first data obtained from a strand of hair acquired from a subject; a second device 200B, 200C configured to acquire second data obtained from a specimen acquired from the subject at a different acquisition time from that of the strand of hair; and a computer 100 configured to: receive the first data from the first device 200, receive the second data from the second device 200B, 200C, and generate a trend of a mental or physical state of the subject based on the first data and the second data.

The embodiments disclosed here are to be understood as being in all ways exemplary and in no ways limiting. The scope of the present disclosure is defined not by the foregoing descriptions but by the appended claims, and is intended to include all changes equivalent in meaning and scope to the claims.

### REFERENCE SIGNS LIST

1 Network system
100 Server
110 Processor (CPU)
120 Memory
121 User database
122 Analysis database
130 Display
140 Operation unit
160 Communication interface
200 First analysis device
200B Second analysis device
200C Third analysis device
200X Analysis device
200Y Server
210 Processor
220 Memory
230 Display
240 Operation unit
260 Communication interface
290 Analysis unit
300 Smartphone
310 Processor (CPU)
320 Memory
330 Display
340 Operation unit
360 Communication interface
380 Speaker
400 Printer
700 Mass database

1. A computer comprising:
a memory configured to store:
first data obtained from a strand of hair acquired from a subject, and
second data obtained from a specimen acquired from the subject, the specimen being different in acquisition time from the strand of hair; and
a processor configured to generate a trend of a mental or physical state of the subject based on the first data and the second data.

2. The computer according to claim 1, further comprising a communication interface,
wherein the processor is configured to acquire the first data and the second data from another device via the communication interface.

3. The computer according to claim 1, further comprising a communication interface,
wherein the processor is configured to:
acquire the first data from a first other device via the communication interface, and
acquire the second data from a second other device via the communication interface.

4. The computer according to claim 1,
wherein the second data is obtained from a strand of hair acquired from the subject, the strand of hair being different in acquisition time from the strand of hair.

5. The computer according to claim 1,
wherein the second data is obtained from a specimen acquired from the subject other than a strand of hair, the specimen being different in acquisition time from the strand of hair.

6. The computer according to claim 1,
wherein the processor is configured to generate a future state of the subject based on data acquired from a subject other than the subject.

7. The computer according to claim 6,
wherein the processor is configured to generate advice for the future state.

8. The computer according to claim 1,
wherein the processor is configured to:
transmit, to the subject when a first predetermined condition is satisfied, information including the trend via the communication interface, and
transmit, to a predetermined institution when a second predetermined condition is satisfied, information including the trend via the communication interface.

9. An information processing method in a computer, the information processing method comprising:
acquiring, by a processor, first data obtained from a strand of hair acquired from a subject;
acquiring, by the processor, second data obtained from a specimen acquired from the subject, the specimen being different in acquisition time from the strand of hair; and
generating, by the processor, a trend of a mental or physical state of the subject based on the first data and the second data.

10. A network system comprising:
a device configured to acquire:
first data obtained from a strand of hair acquired from a subject, and
second data obtained from a specimen acquired from the subject, the specimen being different in acquisition time from the strand of hair; and
a computer configured to:
receive the first data and the second data from the device, and
generate a trend of a mental or physical state of the subject based on the first data and the second data.

11. (canceled)

12. The network system according to claim 10, wherein
the device comprises
a first device configured to acquire first data obtained from a strand of hair acquired from a subject;
a second device configured to acquire second data obtained from a specimen acquired from the subject, the specimen being different in acquisition time from the strand of hair; and
the computer is configured to:
receive the first data from the first device,
receive the second data from the second device, and
generate a trend of a mental or physical state of the subject based on the first data and the second data.
